(19) United States
(12) Patent Application Publication
(10) Pub. No.: US 2025/0204842 A1
(43) Pub. Date: Jun. 26, 2025

(54) TECHNIQUES FOR MEASURING ATYPICAL NEURODEVELOPMENT IN NEONATES BASED ON AUDITORY BRAINSTEM RESPONSE (ABR) TESTS

(71) Applicant: Rutgers, The State University of New Jersey, New Brunswick, NJ (US)

(72) Inventor: Elizabeth B. TORRES, Piscataway, NJ (US)

(21) Appl. No.: 18/869,522

(22) PCT Filed: Jun. 1, 2023
(86) PCT No.: PCT/US23/67729

§ 371 Date: Nov. 26, 2024

Related U.S. Application Data
(60) Provisional application No. 63/347,809, filed on Jun. 1, 2022.

(51) Int. Cl. A61B 5/38; A61B 5/00; A61B 5/372; G16H 50/20; G16H 50/70

(52) U.S. Cl. CPC A61B 5/38; A61B 5/372; A61B 5/4005; A61B 5/4058; G16H 50/20; G16H 50/70

(57) ABSTRACT

Techniques for determining a condition of a subject includes recording auditory brain stem (ABS) electrical signal data in a subject in response to an auditory signal delivered to the subject. First data is determined, wherein the first data indicates a temporal latency for each vertex in a set of one or more vertices in the ABS electrical signal or a property of one or more micropeaks in a portion of the electrical signal away from the vertices, or some combination. Each vertex indicates a response from a different anatomical location from the cochlea to the auditory cortex of the subject. A condition of the subject is determined based on a distance of the temporal latency determined for each vertex, or the property of the micropeaks, or some combination, from a predetermined set of values for temporal latency or property of micropeaks in a population of control individuals.

### CROSS-REFERENCE TO RELATED APPLICATION

This application claims priority to PCT Application No. PCT/US23/67729, filed Jun. 1, 2023, which claims the benefit of U.S. Provisional Patent Application No. 63/347,809, filed Jun. 1, 2022, under 35 U.S.C. § 119, which is incorporated herein by reference in its entirety.

### BACKGROUND

An Auditory Brainstem Response (ABR) test measures the reaction of the parts of a child's nervous system that affect hearing. The ABR test measures the hearing nerve's response to sounds and is a routine test for neonates, from premature babies to neonates up to six months in age to children up to seven years in age. The ABR test is typically performed when the child is sleeping or lying perfectly still, relaxed and with his or her eyes closed, and may involve anesthesia. Three to four electrodes are placed on a child's head and in front of his or her ears and connected to a computer. As sounds are introduced through earphones over the child's cars, the electrodes measure how the child's auditory nerves respond to them.

According to Wikipedia, “The measured recording is a series of six to seven vertex positive waves of which I through V are evaluated. These waves, labeled with Roman numerals in Jewett and Williston convention, occur in the first 10 milliseconds [(ms)] after onset of an auditory stimulus. The ABR is considered an exogenous response because it is dependent upon external factors. When interpreting the ABR, we look at amplitude (the number of neurons firing), latency (the speed of transmission), interpeak latency (the time between peaks), and interaural latency (the difference in wave V latency between cars). The ABR represents initiated activity beginning at the base of the cochlea and moving toward the apex over a 4 ms period of time. The peaks largely reflect activity from the most basal regions on the cochlea because the disturbance hits the basal end first and by the time it gets to the apex, a significant amount of phase cancellation occurs. The ABR is used for newborn hearing screening, auditory threshold estimation, intraoperative monitoring, determining hearing loss type and degree, and auditory nerve and brainstem lesion detection, and in development of cochlear implants.”

### SUMMARY

Techniques are provided for using ABR test results to detect differences in neurodevelopment in neonates and diagnose any neurodegenerative conditions, such as autism spectrum disorder (ASD).

In a first set of embodiments, a method for determining a condition of a subject includes: recording auditory brain stem (ABS) electrical signal data in a subject in response to an auditory signal delivered to the subject. The method also includes determining first data that indicates a temporal latency for each vertex in a set of one or more vertices in the ABS electrical signal. Each vertex indicates a response from a different anatomical location from the cochlea to the auditory cortex of the subject. The method further includes determining a condition of the subject based on a distance of the temporal latency determined for each vertex from a predetermined set of values for temporal latency in a population of control individuals.

In some embodiments of the first set, the population of control individuals includes a plurality of populations of control individuals, each population having a different set of values for temporal latency and associated with a different neurodevelopmental condition. In some of these embodiments, said recording auditory brain stem (ABS) electrical signal data in the subject includes recording auditory brain stem (ABS) electrical signal data in the subject in response to a plurality of auditory signals delivered to the subject. The first data indicates a distribution of latencies for each vertex over the plurality of auditory signals delivered to the subject. The distance is a distance between the distribution of latencies of the subject to the distributions of latencies in the plurality of populations of control individuals.

In a second set of embodiments, a method for determining a condition of a subject includes recording auditory brain stem (ABS) electrical signal data as in the first set of embodiments. In this set, however, the method includes instead determining first data that indicates a property of micropeaks in the ABS electrical signal. The micropeaks occur in a first portion of the ABS electrical signal away from a different second portion that includes a set of one or more vertices, wherein each vertex indicates a response from a different anatomical location from the cochlea to the auditory cortex of the subject. In this second set of embodiments, the method further includes instead determining a condition of the subject based on a distance of the property of the micropeaks from a predetermined set of values for the property of the micropeaks in a population of control individuals.

In some embodiments of the second set, the population of control individuals includes a plurality of populations of control individuals, each population having a different set of values for the property of the micropeaks and associated with a different neurodevelopmental condition. In some of these embodiments, the first data indicates a distribution of the property for each micropeak over a plurality of micropeaks; and the distance is a distance between the distribution of the property of micropeaks in the subject to the distributions of the property in the plurality of populations of control individuals.

In other sets of embodiments, a non-transitory computer readable medium, or an apparatus, or a system is configured to perform one or more of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a block diagram that depicts an Auditory Brainstem Response (ABR) test setup, according to an embodiment;

FIG. 1B is a graph that illustrates example vertices in an electrical measurement from an ABR test setup, according to an embodiment;

FIG. 1C is a block diagram that illustrates anatomical features associated with the vertices, according to an embodiment;

FIG. 1D is a graph that illustrates an example electrical measurement from an ABR test setup spanning three sets of vertices from three different audio stimulations, according to an embodiment;

FIG. 2A through FIG. 2H are graphs and a table that illustrate example neurodevelopmental differences in distributions of latencies of vertices in electrical measurements from the ABR test setup, according to the experimental embodiment;

FIG. 3A is a flow diagram that illustrate an example method to detect neurodevelopmental condition of a subject based on latencies of vertices, according to an embodiment;

FIG. 3B is a graph that illustrates example features of a latency PDF that show differing temporal development in different populations, according to an embodiment;

FIG. 4A is a graph that illustrates example electrical measurement around a set of vertices from an ABR test setup, according to an embodiment;

FIG. 4B is a graph that illustrates example properties of a primary peak in the electrical measurements associated with a set of vertices, according to an embodiment;

FIG. 4C and FIG. 4D are graphs that illustrate example properties of micro-peaks in electrical measurements apart from the primary peak, according to an embodiment;

FIG. 4E is a graph that illustrates example normalized peaks in electrical measurements from an ABR test setup, according to an embodiment;

FIG. 5A and FIG. 5B are graphs that illustrate example distributions of inter-micropeak latencies in electrical measurements from an ABR test setup, which detect differences in neurodevelopment, according to an embodiment;

FIG. 5C and FIG. 5D are graphs that illustrate example distributions of prominences of micropeaks in electrical measurements from an ABR test setup, which detect differences in neurodevelopment, according to an embodiment;

FIG. 6 is a flow diagram that illustrate an example method to detect differences in neurodevelopment of a subject based on properties of peaks in electrical measurements from the ABR test setup, according to an embodiment;

FIG. 7 is a table that summarizes ABR test subjects, according to an experimental embodiment;

FIG. 8A and FIG. 8B are graphs that illustrate example neurodevelopmental differences in parameters of distributions of latencies of vertices, according to the experimental embodiment;

FIG. 9A through FIG. 9D are graphs that illustrate example neurodevelopmental differences in distributions of latencies of vertices, according to the experimental embodiment;

FIG. 10 is a three-dimensional graph that illustrate example clear separation by neurodevelopmental differences in empirical distributions of prominences of peaks in electrical measurements from the ABR test setup, according to the experimental embodiment;

FIG. 11 is a block diagram that illustrates a computer system ˜00 upon which an embodiment of the invention may be implemented;

FIG. 12 is a block diagram that illustrates a chip set upon which an embodiment of the invention may be implemented;

FIG. 13 is a block diagram of exemplary components of a mobile terminal (e.g., cell phone handset) for communications, which is capable of operating in the system of FIG. 1A, according to one embodiment.

### DETAILED DESCRIPTION

Techniques are described to detect or quantify differences in neurodevelopment in neonates and diagnose any neurodegenerative conditions. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of detecting Autism Spectrum Disorder (ASD) in neonates. However, the invention is not limited to this context. In other embodiments, the same or different neurodevelopmental disorders are detected or quantified in utero, or in neonates, or in young children up to about 7 years of age, or some combination. A variety of neurodevelopmental disorders are expected to be detected or quantified using these methods including language related disorders, cerebral palsy and a variety of disorders incorporated by the term ASD such as Attention Deficit/hyperactivity Disorder (ADHD), sensory disorders, and others under the Diagnostic and Statistical Manual of Mental Disorders, Fifth Edition (DSM V) criteria.

### 1. Overview

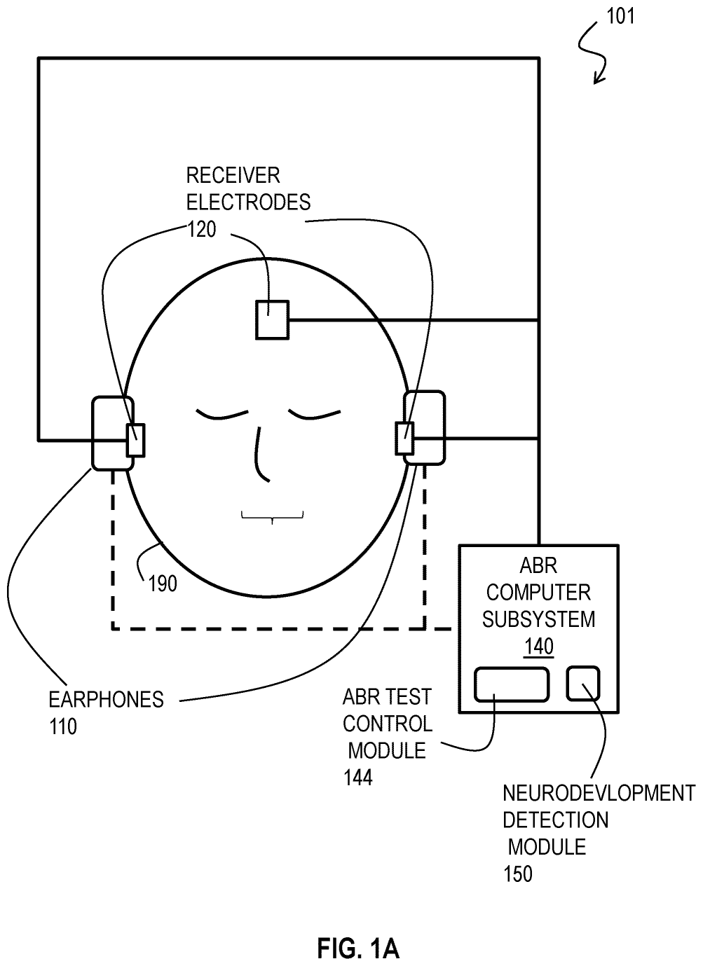

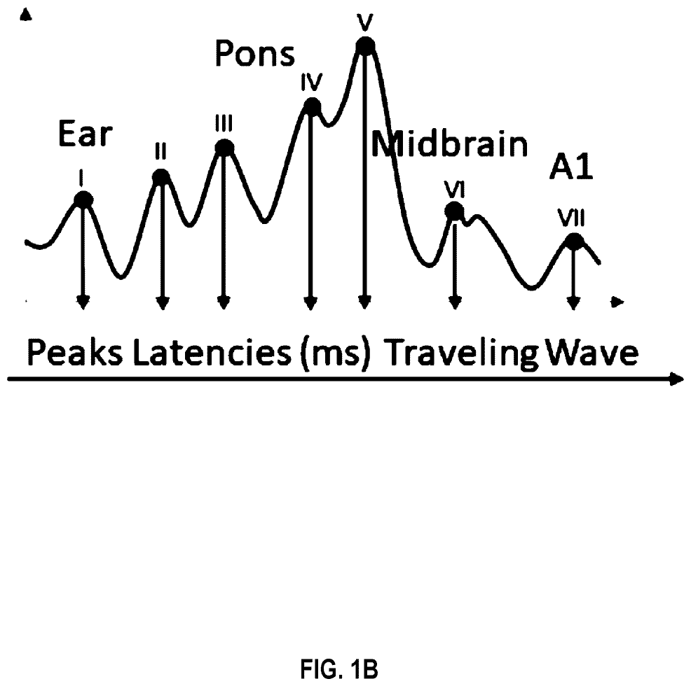

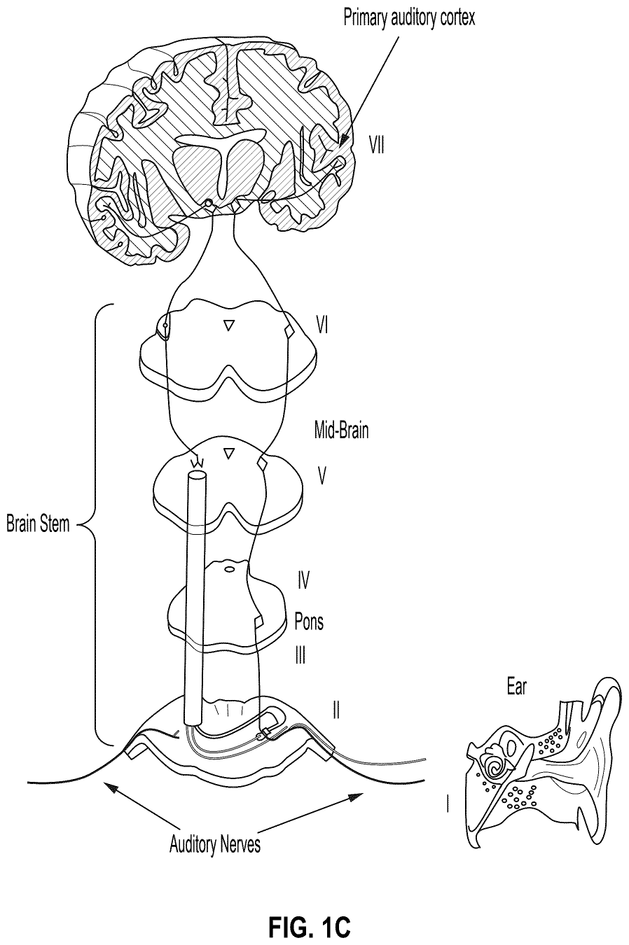

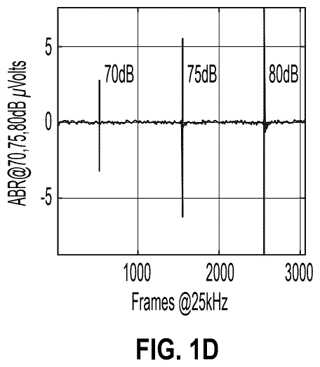

FIG. 1A is a block diagram that depicts an Auditory Brainstem Response (ABR) test system 101, according to an embodiment. Although a head of a subject 190 is depicted, the subject 190 is not part of the system 101. The system includes earphones or other speakers 110 that are configured to be placed over the ears of a subject and deliver sound to the ears of the subject 190. The system includes an ABR computer subsystem 140 in communication with the earphones 110 by wired or wireless connections depicted as dashed lines. The system also includes one or more electrodes 120 configured to be placed at corresponding locations on the skin of the head of the subject 190 to pick up electrical signals and transmit them to the ABR computer subsystem 140 by wired or wireless connections depicted as solid lines. The ABR computer subsystem includes hardware and software configured to perform as an ABR test control module 144. The function of the control module 144 is to operate the earphones or speakers to deliver an auditory signal such as one or more tones of a predetermined frequency and volume. The function of the control module 144 is also to record on a computer-readable medium or display, or both, the electrical signal detected at each electrode 120.

According to various embodiments, the system 101 includes a neurodevelopment detection module 150 comprising hardware and or software and configured to detect attributes of the electrical signal from electrodes 120 that can be used to infer a condition of the subject 190 by comparing those electrical signals to electrical signals associated with one or more populations of individuals with known neurodevelopmental conditions. Module 150 is configured to implement one or more of the methods described herein. Each of modules 144 and 150 include one or more data structures (not shown explicitly) to hold data. For example, module 144 includes one or more data structures that hold data that indicates the auditory signals to be delivered to earphones 110. For example, module 150 includes one or more data structures that hold data that indicate electrical signals received from electrodes 120, or distribution of properties of electrical signals associated with one or more populations, or some combination.

Although processes, equipment, and data structures are depicted in FIG. 1A as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts.

FIG. 1B is a graph that illustrates example vertices in an electrical measurement from an ABR test system, according to an embodiment. The electrical measurement received from each electrode includes a plurality of vertices received during a time interval of a few milliseconds after an auditory signal is delivered to the subject. Each vertex is a local maximum in the electrical signal and typically five to seven vertices are identified after an auditory signal is delivered to the subject. The various vertices are designated by Roman numbers I, II, III, IV, V, VI, VII, from earliest to latest, respectively. The different vertices are associated with neural activity at different anatomical locations along the nerve path from cochlea in the inner ear of the subject to the main auditory cortex of the brain of the subject. FIG. 1C is a block diagram that illustrates anatomical features associated with the vertices, according to an embodiment. Vertex I is associated with the cochlea of the inner ear, vertex II with the auditory nerves, vertices III and IV with the Pons in the brainstem, vertex V with the Mid-brain of the brainstem, vertex VI with the top of the brainstem and vertex VII with the primary auditory cortex of the brain itself. At different electrodes, different vertices may have different heights and the same vertices may arrive at different times.

Referring again to FIG. 1B, the difference in time from the time that the auditory signal is delivered until the time a vertex is detected at an electrode is called the full latency of that vertex. In some embodiments, the time difference between the first vertex and any other vertex is called the relative latency. In some embodiments, the time between successive vertices is called the interpeak latency.

FIG. 1D is a graph that illustrates an example electrical measurement from an ABR test setup spanning three sets of vertices from three different audio stimulations, according to an embodiment. The horizontal axis indicates time in frames of 1/25000 of a second, so that one millisecond corresponds to 25 frames. There is an electrical measurement at each frame from at least one electrode. The vertical axis indicates the strength of the electrical signal voltage in microVolts (μV, 1 μV=10⁻⁶ volts). A few milliseconds after each auditory stimulation, there is a large excursion of voltage corresponding to the arrival of one or more vertices. Each auditory stimulation has a different acoustic volume, indicated by decibel (dB) level. The first auditory signal is at 70 dB and elicits a first voltage response in the electrical signal near the 500^{th} frame. The second auditory signal is louder at 75 dB and elicits a larger second voltage response in the electrical signal near the 1500^{th} frame. The third auditory signal is loudest at 80 dB and elicits a largest third voltage response in the electrical signal near the 2500^{th} frame. The time of the auditory signal is not shown but is presumably within about 10 milliseconds (250 frames) of the electrical response.

During an ABR test, the auditory signals and electrical response shown in FIG. 1D are repeated multiple times, typically 10 or more. The large electrical excursions and the associated vertices are reviewed to determine that the subject has a normal or abnormal hearing response appropriate for the age of the fetus, neonate or child.

In various embodiments described below, other features of the electrical signal of FIG. 1D are processed to detect or otherwise quantify the neurodevelopment condition of the subject beside hearing acuity.

### 1.1 Latency as Indication of Neurodevelopmental Condition

The development of the brainstem monoaminergic circuitry is fundamental to later cortical development. It is also essential for the tonotopic organization of the auditory system enabling it to receive sensory-evoked signals and produce orderly measurable responses. As such, early disruptions in the brainstem have been hypothesized to play a causal role in neurodevelopmental derail, inevitably leading to the umbrella-type autism spectrum disorders diagnosis. Yet, how disturbances at different levels may lead to differences in latencies as signals propagate through different brainstem region has not heretofore been quantified.

In a rare opportunity, we had access to data from three distinct click levels at 70, 75 and 80 dB (see methods), to systematically track the propagation times of the response waveform and assess the intactness of the cranial nerve. We used seven standard regions of interest, a subset of them previously characterized in neonates [29]. They are depicted in FIG. 1B. Yet, we did not average these values across the pool of neonates under consideration, using an a priori assumed theoretical distribution (as in e.g., [29]). Instead, we empirically estimated the best continuous family of probability density functions PDFs fitting these data. We found distributions of latencies with heavy tails.

The distribution of latencies was found to vary with neurodevelopmental categories. For example, when a group of individuals was divided into a first population showing normal neurodevelopment and a second population that were eventually diagnosed with autism spectrum disorder (ASD), differences in the distribution of latencies were evident. The different empirical distributions were represented by different probability density functions for different neurodevelopmental conditions.

Various empirical distributions are well fit by the continuous Gamma family of probability distributions using maximum likelihood estimation (MLE). As such, empirically obtaining the Gamma shape and scale parameters and plotting them on the Gamma parameters' plane with 95% confidence intervals, provided an overall view of the stochastic signatures of the different groups of neonates as latencies propagated across brainstem regions I-VII. Furthermore, the Gamma PDFs thus obtained for each set of latency values revealed fundamental shifts in stochastic signatures between the babies who received the autism diagnosis and those who did not.

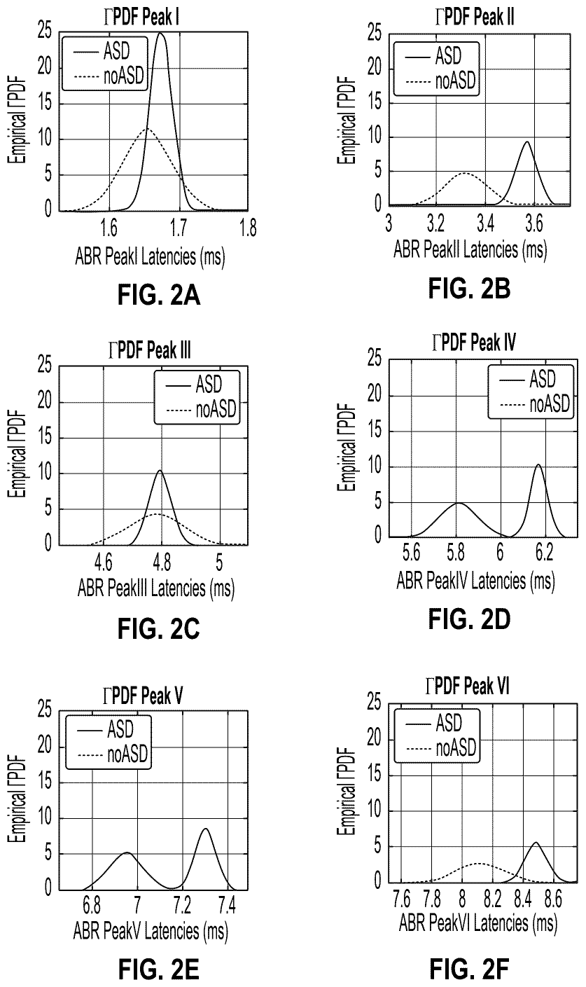

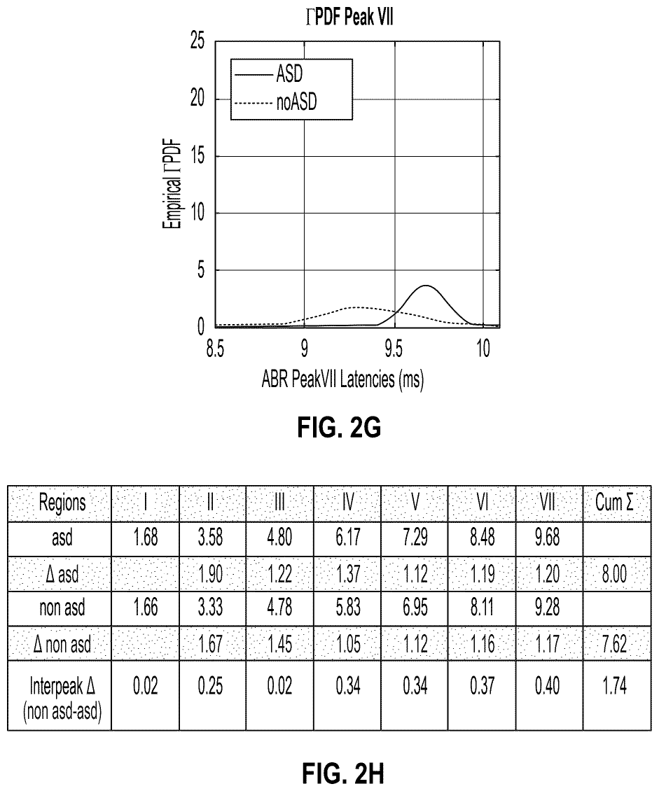

FIG. 2A through FIG. 2H are graphs and a table that illustrate example neurodevelopmental differences in distributions of latencies of vertices in electrical measurements from the ABR test setup, according to the experimental embodiment. Note that the vertices are called “peaks” in the drawings but are called vertices in this specification in order to avoid confusion with primary peaks and micropeaks described in more detail below. In general, a Roman numeral labeled “peak” is one of the seven vertices described herein. In each of FIG. 2A through FIG. 2G, the horizontal axis indicates latency of arrival of one of the seven vertices, labeled on each axis, from vertex I through vertex VII, respectively. In each of FIG. 2A through FIG. 2G, the vertical axis indicates the number of occurrences of peaks at a specified latency. In each graph, one trace indicates the Gamma function fit to latencies in a population showing normal neural development and a second trace, labeled ASD, indicates the Gamma function fit to latencies .in a population eventually diagnosed with ASD.

The graphs clearly demonstrate the (cumulative) delays (longer latencies) in the ASD group compared to the non-ASD group along with the narrower spread of values in the ASD group.

FIG. 2H is a table that shows vertex latencies for each site in the two cohorts and inter-vertex latency differences. The last column shows the cumulative sum of the delays and makes evident the appreciable ASD neonates' delay of 1.74 ms (especially considering that sound processing is generally agreed to be on a microsecond time scale). The cumulative effect across these sites of the brainstem revealed 1.74 ms difference, with 0.40 ms as the longest local delay in the vertex VII, while vertex III had the shortest delay 0.05 ms. The asd neonates show a net cumulative delay of 8 ms vs. 7.62 ms in the non-asd neonates.

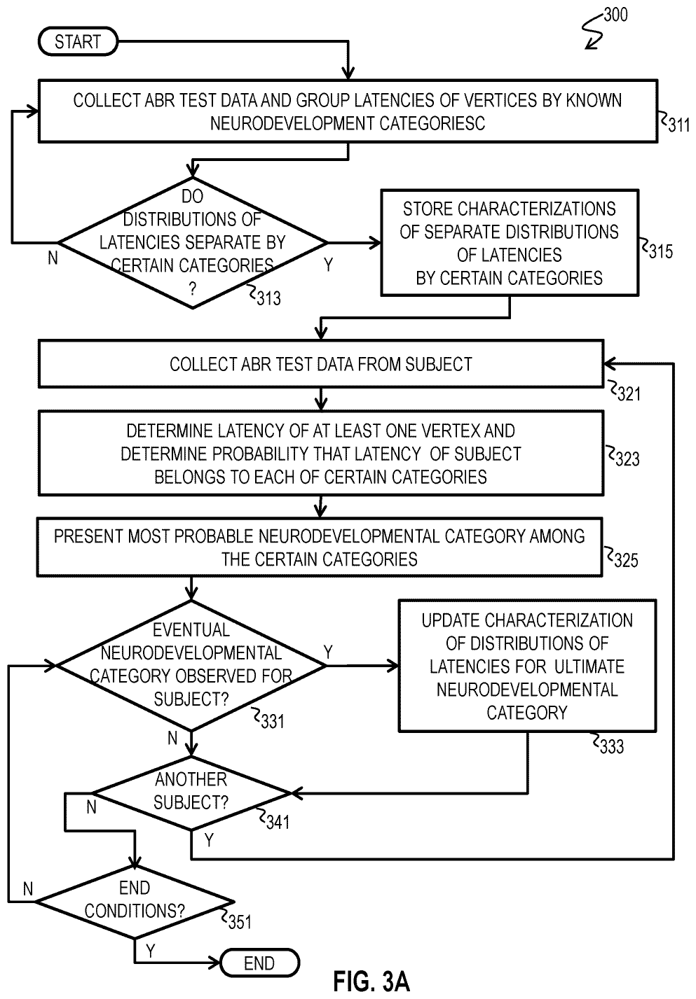

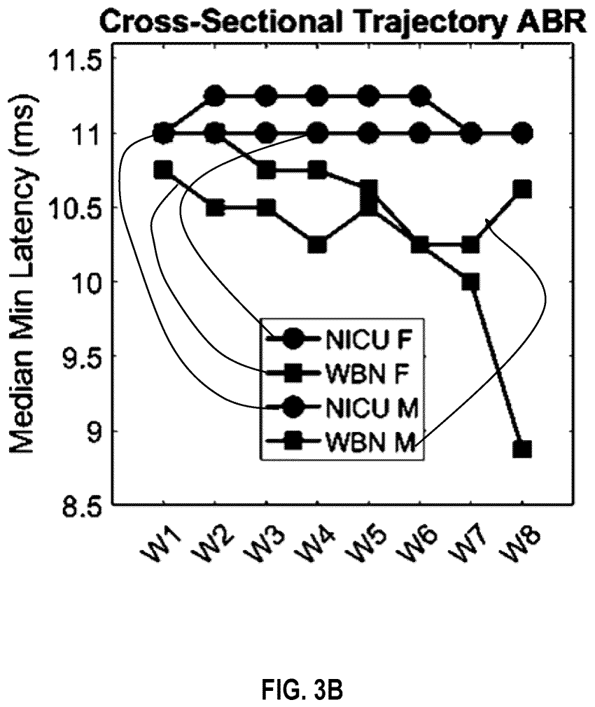

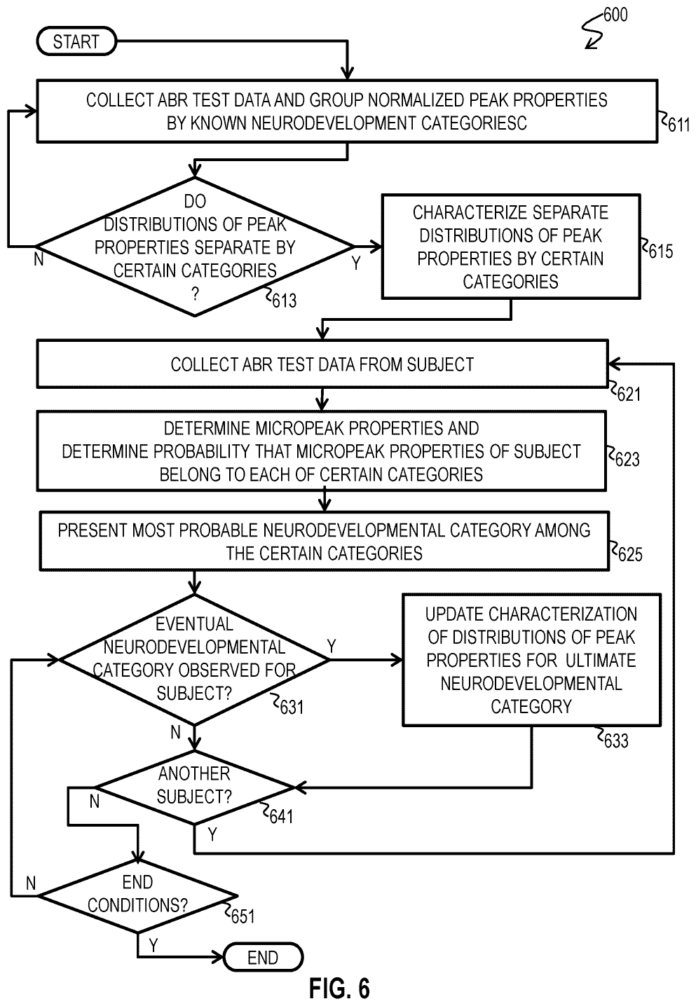

In general, differences in latencies are expected for other neurodevelopmental conditions or disorders. FIG. 3A is a flow diagram that illustrate an example method 300 to detect neurodevelopmental condition of a subject based on latencies of vertices, according to an embodiment. Although steps are depicted in FIG. 3A, and in subsequent flowchart FIG. 6, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 311, ABR test data is collected for a plurality of individuals at various ages, from fetal ages to premature ages to neonates and on into childhood. Each individual is further tracked and eventually assigned to one of several neurodevelopmental categories, each reflective of a certain neurodevelopmental condition, such as typical or ASD, among others. For each neurodevelopmental category, the distribution of latencies of each of one or more of the seven vertices is determined. Any latency can be used including the full latency (travel time), the relative latency, or the inter-vertex latency, or some combination. For example, an empirical histogram of latencies is determined for each of one or more vertices, or a functional fit, such as a Gamma function fit, to the empirical histogram is performed, and the values of the functional fitting parameters are stored for each vertex and neurodevelopmental category.

In step 313, it is determined whether there are two or more categories with statistically significant differences in distributions. Any method may be used to determine the statistical significance of differences in two distributions. Example methods include the Kolmogorov-Smirnov test, the Jensen-Shannon divergence, which is an extension of the Kullback-Leibler divergence (one sided), the Wasserstein distance (the Earthmover's distance, EMD) and there an equivalent UNIFRAC distance used in the microbiome world, and classical methods for non-normal cases such as the Kruskal-Wallis test (non-parametric ANOVA1) or Friedman test (ANOVA2), and the two-sided Wilcoxon rank sum test, among others. If not, control flows back to step 311 to continue to collect ABR test data for different categories of neurodevelopment. If a significant difference is found among two or more distributions of certain categories, then control passes to step 315.

In step 315, the certain categories of neurodevelopmental conditions are stored along with data indicating their distributions, such as an empirical histogram, or mean and variance and skewness of an empirical histogram, or the values of one or more functional fitting parameters, such as values for shape and scale of the Gamma Function, or some combination. Any method may be used to store this information, in one or more data structures, such as a flat file or files or a relational database. These are the predetermined distributions of certain neurodevelopmental categories, such as the distribution depicted in FIG. 2A through FIG. 2G for typical and ASD categories, or the plots of shape and scale parameter values as described in more detail in the examples section, below.

In step 321, ABR test data is collected from a subject, such as during a routine clinical visit for testing hearing acuity of a neonate. In some embodiments, the collection of step 321 is initiated for other reasons, e.g., explicitly to determine the neurodevelopmental conditions of the subject. Step 321 includes a sufficient number of auditory stimulation and electrical measurements to obtain a good mean value of latency for one or more of the seven vertices in the electrical signal. In some embodiments, step 321 includes a sufficient number of auditory stimulation and electrical measurements to obtain a good representation of the distribution of latencies for each of one or more of the seven vertices in the electrical signal.

In step 323, the latency, or latencies for various vertices, or the distribution of latencies for one vertex, or the distributions of latencies for various vertices, are compared to the predetermined latencies stored for the certain neurodevelopmental categories, and the probability that the lat4ency or distribution belongs to each neurodevelopmental category is determined. Any method may be used to determine this. For example, the z-test or t-test or Anderson-Darling statistic or a similarity statistic, or Earth-movers distance can be used, among others, listed above. In some embodiments, machine learning (ML) methods are used. For example, a leave one out decoder method is used, in which the classifier is trained on all training sets except one left out to be classified. Repeating leaving out a different one, distributions of the correct versus errors are built to obtain confusion matrices. The automatic classification power of the data is then evaluated. Other ML methods are also amenable Because the PDFs are so different (no matter what combination of babies-trials you take from each group) these classifiers do a great job at automatically separating the babies correctly by label (ASD vs no ASD).

In step 325, the most probable neurodevelopmental category is presented, e.g., to the doctor, clinician or other caregiver. For example, the category is presented on a computer display. In some embodiments, the presentation includes the probability of the category. In some embodiments, several categories and associated probabilities are displayed. This serves to notify the doctor, clinician or other caregiver of the usefulness of further testing, observation, or treatment for the subject. For example, the data can be presented like a growth chart currently used in pediatrics to track the baby's development across wellness visits. Here, the PDFs are estimated, and the chart is built with PDF parameters values per age, so the pediatrician knows if something is wrong across visits, e.g., by splitting the data per week for 8 weeks. FIG. 3B is a graph that illustrates example features of a latency PDF that show differing temporal development in different populations, according to an embodiment. This is comparing neonatal intensive care unit (NICU) vs Well Baby Nursery (WBN), using clear peaks available for vertices III and V. Different temporal trajectories are obtained for different parameters, but FIG. 3B is based on the minimum latency of the waveform. One can tell which babies are stunted because the latencies are not decreasing. One gets different trajectories for different signal vertices and different PDF parameters. In general, WBN neonates are trending down in latency, but NICU are steady, not changing much for either females or males.

In some embodiments, the stored data is updated as the subject's eventual development is tracked. In step 331, it is determined whether the eventual neurodevelopmental category is observed for the subject. For example, it is determined that the subject undergoes typical neurodevelopment. If not, control passes to step 341 to determine if there is another subject. However, if the eventual category is observed for the subject, then control first passes to step 333 and then to step 341. In step 333, the characterization of the latency or distributions of latencies for the neurodevelopmental category of the subject previously stored during step 315 is updated during step 333. The observed latencies for the current subject at the various ages or stages of development are added to the stored measurements at those ages or stages. Thus, the system eventually learns a more comprehensive characterization of the neurodevelopmental category.

In step 341, it is determined whether there is another subject for an ABR test. If so, control passes back to step 321 and following, described above. If not, control passes to step 351.

In step 351, it is determined if end conditions are satisfied, e.g., that the system is to be powered down or to halt tracking the development of any subjects. If not, control passes back to step 331, described above. If so, then the process ends.

### 1.2 Micropeaks as Indication of Neurodevelopmental Condition

Electrical measurements are collected before or after each auditory stimulation, or both, during an ABR test. Statistics of fluctuations in these measurements, called micropeaks, have been found to indicate neurodevelopmental conditions. Thus, according to some embodiments, micropeaks are used to indicate neurodevelopmental condition.

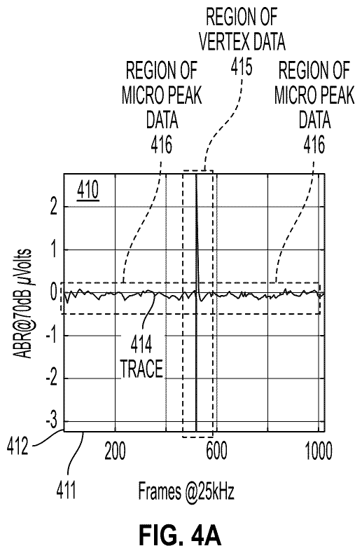

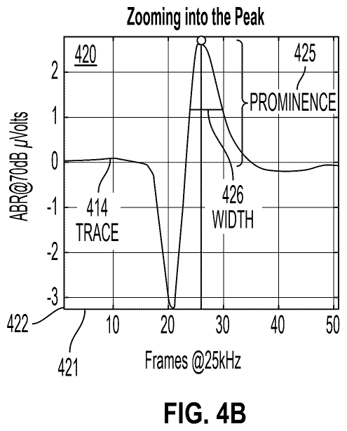

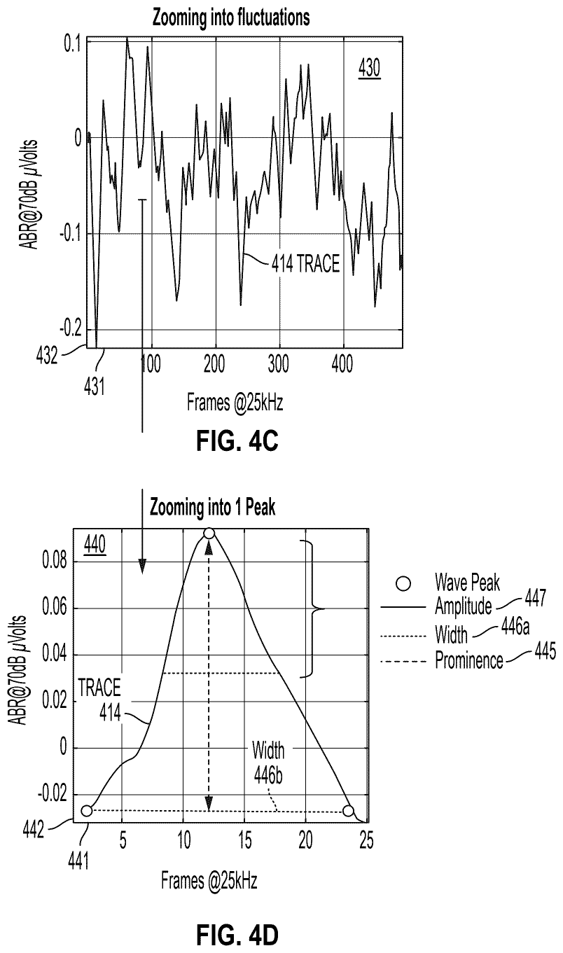

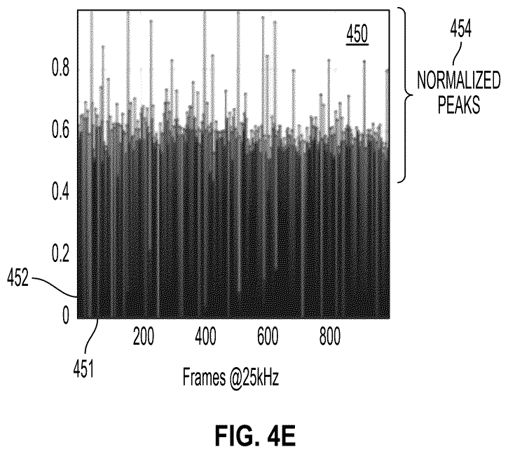

FIG. 4A is a graph that illustrates example electrical measurement around a set of vertices from an ABR test setup, according to an embodiment. The horizontal axis indicates frames at 25 kilHertz (kHz, 1 kHz=10³ cycles per second), thus 25 frames correspond to one millisecond. The vertical axis indicates electrical signal amplitude in μV. Trace 414 indicates electrical measurements collected over 40 milliseconds around an auditory stimulation with a volume of 70 dB at a time not shown on the plot. One temporal region, or portion, 415 of the trace 414 spans the large electrical signals of the vertices described above, at about 550 ms. The remaining temporal region, or portion, 416 includes only smaller fluctuations called micropeaks. One or more properties of these micropeaks indicate background neural activity and have been found to distinguish various neurodevelopmental conditions.

FIG. 4B is a graph that illustrates example properties of a primary peak in the electrical measurements associated with a set of vertices, according to an embodiment. The horizontal axis indicates frames at 25 kHz and extends for 50 frames, thus represents two milliseconds (ms, 1 ms=10⁻³ seconds). The vertical axis indicates electrical amplitude in μV. The portion of trace 414 in this region 415 represents the subject's electrical response to the auditory stimulation. The trace 414 indicates essentially a correlation with the multiple vertices I to VII expected in this electrical signal and thus the primary peak represents a Dirac delta function at the time of receipt of the expected signal. The location of the primary peak is considered to be at frame 25 (1 ms along the axis) and the properties of this primary peak include a prominence 425 of about 2.5 mV above a zero electrical amplitude and a width 426 at half height of about 6 frames (about 240 microseconds).

FIG. 4C and FIG. 4D are graphs that illustrate example properties of micro-peaks in electrical measurements apart from the primary peak, according to an embodiment. For each graph, the horizontal axis indicates a number of frames at 25 kHz, and the vertical axis indicates the electrical amplitude in μV for data collected around the peak response to an auditory signal amplitude of 70 dB. This portion of trace 414 include multiple fluctuations. A micropeak is considered to be the maximum electrical value between successive minimum electrical values. FIG. 4C shows multiple micropeaks along a portion of trace 414. FIG. 4D shows a single micropeak from FIG. 4C. The single micropeak in FIG. 4D at frame 12 along the horizontal axis has properties that include a prominence 445 indicated by a dashed line segment from the largest minimum value on each side of the peak to the maximum value, an amplitude of half the prominence value, a width 446a at half height, and a width 446b at the base which is at the voltage of the maximum of the two minima bracketing the micropeak.

In some embodiments the micropeaks in the signal are normalized by the value of the largest prominence of all the micropeaks in the ABR electrical signal being processed. In some embodiments, the normalization of any peak, including the primary peak, is computed using Equation 1.

$$P {n}^{*} = Pn / [ Pn + Avg ( Pmin 1 , Pmin 2 ) ] \qquad ( 1 )$$

Where Pn is the prominence of the nth peak, Pn* is the normalized prominence of the nth peak, and Avg (Pmin1, Pmin2) is the average of all the points between the two local minima Pmin1 and Pmin2, including the local maximum (micropeak) Pn When the Avg (Pmin1, Pmin2) is small compared to the prominence of the nth peak, such as for the primary peak, the value of Pn* approaches 1. Such normalization tends to scale out allometric differences in length, weight, anatomical size, and other characteristics for individuals in the same group (e.g., same age or stage of development or neurodevelopmental condition or combination)

The normalized prominences of the micropeaks and primary peak are plotted in FIG. 4E. FIG. 4E is a graph that illustrates example normalized peaks in electrical measurements from an ABR test setup, according to an embodiment. The horizontal axis indicates frame at 25 kHz, and the vertical axis indicates normalized prominences of micropeaks. Each micropeak is plotted at the frame where it occurs with a normalized prominence value between 0 and 1.

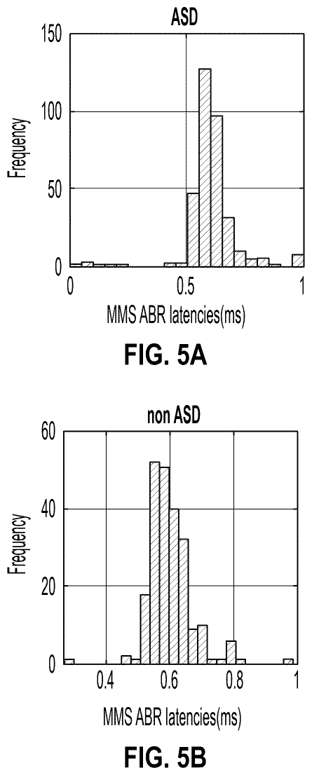

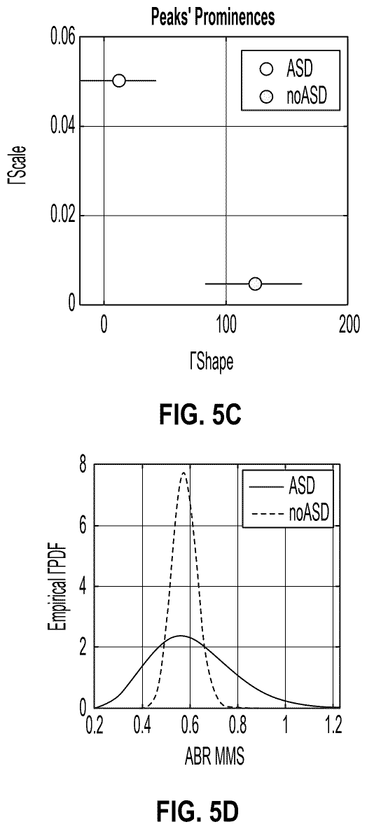

Similar normalization can be performed for other properties, such as interpeak latencies, so that the primary peak can be combined with the micropeaks. FIG. 5A and FIG. 5B are graphs that illustrate example distributions of inter-micropeak latencies in electrical measurements from an ABR test setup, which detect differences in neurodevelopment, according to an embodiment. For both graphs, the horizontal axis indicates 0.05 ms bins of inter-micropeak latency from 0 to 1 ms; and the vertical axis indicates occurrences. FIG. 5A depicts the empirical distribution of micropeak latencies for the population of individuals diagnosed with ASD, and FIG. 5B depicts the empirical distribution of micropeak latencies for the non-ASD population. There is a clear difference, with the ASD population showing long latencies and a narrower distribution.

FIG. 5C and FIG. 5D are graphs that illustrate example distributions of prominences of micropeaks in electrical measurements from an ABR test setup, which detect differences in neurodevelopment, according to an embodiment. FIG. 5C shows the Gamma function parameters that fit the empirical distributions of micropeak normalized prominences. For both ASD and non-ASD populations. The horizontal axis indicates the values of the Gamma shape parameter from 0 to 200. The vertical axis indicates the values of the Gamma scale parameter from 0 to 0.06. The ASD population parameter values clearly separate from the non-ASD population parameter values, even considering the uncertainty bars plotted around each point. FIG. 5D shows the probability density functions corresponding to the Gamma parameter values plotted in FIG. 5C. The horizontal axis indicates normalized micropeak prominences and the vertical axis indicates probability density in relative units. The ASD population shows a narrower and more symmetric range of normalized prominences than the non-ASD population.

In general, differences in micropeak properties are expected for other neurodevelopmental conditions or disorders. FIG. 6 is a flow diagram that illustrates an example method to detect differences in neurodevelopment of a subject based on properties of peaks in electrical measurements from the ABR test setup, according to an embodiment.

In step 611, ABR test data is collected for a plurality of individuals at various ages, from fetal ages, to premature ages, to neonates, and on into childhood. Each individual is further tracked and eventually assigned to one of several neurodevelopmental categories, each reflective of a certain neurodevelopmental condition, such as typical or ASD, among others. For each neurodevelopmental category, the distribution of one or more properties of micropeaks or the primary peak or some combination is determined. Any property can be used including the prominence, the amplitude, the width at half height, the width at base, or the inter-peak latency, or some combination. For example, an empirical histogram of one or more micropeak or primary peak properties is determined for each of one or more peak properties, or the mean, variance and skewness and kurtosis or some combination of the empirical distributions are determined, or a functional fit, such as a Gamma function fit, to the empirical histogram is performed, and the values of the functional fitting parameters are stored for each peak property and neurodevelopmental category.

In step 613, it is determined whether there are two or more categories with statistically significant differences in distributions. Any method may be used to determine the statistical significance of differences in two distributions. Example methods include the Kolmogorov-Smirnov test, and the Earthmover's distance, among others, as described above. If not, control flows back to step 611 to continue to collect ABR test data for different categories of neurodevelopment. If a significant difference is found among two or more distributions of certain categories, then control passes to step 615.

In step 615, the certain categories of neurodevelopmental conditions are stored along with data indicating their distributions, such as an empirical histogram, or mean and variance and skewness of an empirical histogram, or the values of one or more functional fitting parameters, such as values for shape and scale of the Gamma Function, or some combination. Any method may be used to store this information, in one or more data structures, such as a flat file or files or a relational database. These are the predetermined distributions of certain neurodevelopmental categories, such as the distribution depicted in FIG. 5A through FIG. 5D for typical and ASD categories, or the plots of shape and scale parameter values as described in more detail in the examples section, below.

In step 621, ABR test data is collected from a subject, such as during a routine clinical visit for testing hearing acuity of a neonate. In some embodiments, the collection of step 621 is initiated for other reasons, e.g., explicitly to determine the neurodevelopmental conditions of the subject. Step 621 includes a sufficient number of auditory stimulation and electrical measurements to obtain a good distribution of the properties of micropeaks and the primary peak in the electrical signal.

In step 623, the distributions of one or more peak properties, are compared to the predetermined distributions of peak properties stored for the certain neurodevelopmental categories, and the probability that the distribution belongs to each neurodevelopmental category is determined. Any method may be used to determine this. For example, the z-test or t-test or Anderson-Darling statistic or a similarity statistic, or Earth-movers distance can be used, among others, as described above.

In step 625, the most probable neurodevelopmental category is presented, e.g., to the doctor, clinician or other caregiver. For example, the category is presented on a computer display. In some embodiments, the presentation includes the probability of the category. In some embodiments, several categories and associated probabilities are displayed. This serves to notify the doctor, clinician or other caregiver of the usefulness of further testing, observation, or treatment for the subject. In some embodiments an age chart is presented that gives the one or more features of the PDF for two or more categories at different ages, similar to the chart depicted in FIG. 3B.

In some embodiments, the stored data is updated as the subject's eventual development is tracked. In step 631, it is determined whether the eventual neurodevelopmental category is observed for the subject. For example, it is determined that the subject undergoes typical neurodevelopment. If not, control passes to step 641 to determine if there is another subject. However, if the eventual category is observed for the subject, then control first passes to step 633 and then to step 641. In step 633, the characterization of the distributions of peak properties for the neurodevelopmental category of the subject previously stored during step 615 is updated during step 633. The observed distribution of each peak property for the current subject at the various ages or stages of development are added to the stored measurements at those ages or stages. Thus, the system eventually learns a more comprehensive characterization of the neurodevelopmental category.

In step 641, it is determined whether there is another subject for an ABR test. If so, control passes back to step 321 and following, described above. If not, control passes to step 651.

In step 651, it is determined if end conditions are satisfied, e.g., that the system is to be powered down or to halt tracking the development of any subjects. If not, control passes back to step 631, described above. If so, then the process ends.

### 2. Example Embodiments

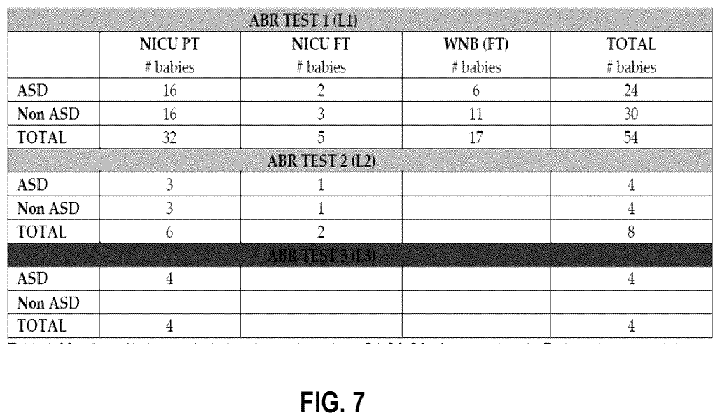

In an example embodiment, the ABR waveforms of 67 neonates were assessed. FIG. 7 is a table that summarizes ABR test subjects, according to an experimental embodiment. The table of FIG. 7 indicates the number of babies included in the study and sets L1, L2, L3 of repeated trials. Each trial contained three levels of sound spanning 70, 75, 80 dB from which the full waveform was saved. The table shows 49 neonates from the neonatal intensive care unit (NICU), (25 full-term (FT, 8 females and 17 males) and 42 pre-term (PT, 15 females and 27 males)), among them, 22 non NICU babies. Of this group, 34 developed along a neurotypical trajectory (17 females and 17 males), but 33 (6 females, 27 males) eventually received the autism diagnosis. These cohorts are named non-asd and asd neonates from here onward.

### 2.1 Latency

In addition to the full waveform data (μV), access to the latency data (ms) from 47 neonates was achieved, with multiple trials in each set of responses to three auditory dB levels. Pooling across all neonates and trials, and using bootstrapping techniques, a larger dataset (above 100 measurements) was built to interrogate fluctuations in latencies and various features of the full waveforms. Furthermore, a personalized approach was used to also examine the individuals in the cohort, in search of self-emerging patterns.

The ABR latencies are compared at millisecond time scale along with the fluctuations in the waveform's primary peak and micropeaks' amplitude (μV) and width (ms) reflecting these responses. Although such waveform's features are seldom examined and the literature remains controversial on their informative power, the methods of FIG. 3A and FIG. 6 were introduced to assess several empirical parameterizations related to the micro-fluctuations of the waveforms' peaks features (amplitude, prominence, and width). These micro-fluctuations are commonly discarded as gross data or smoothed out as noise. Yet, it was found that they automatically separate the asd from the non-asd groups and clearly differentiate across dB levels evoking the responses and differentiating between pre-term (PT) and full-term (FT) neonates.

For latencies of the 7 vertices, the distribution for each group ASD vs noASD comes from pooling all latencies per vertex and then performing comparison of thus obtained PDF. Equal number of instances were included in each group type, (here in this data set each group has different number of participants and different number of trials per participant) by cycling through the dataset, so all possible instances are visited at least once. A distribution of these p-values from multiple comparisons of these two sets gives away the differences, but each pair comparison also gives it away, since they are two different distributions altogether A2), and the two-sided Wilcoxon rank sum test is used.

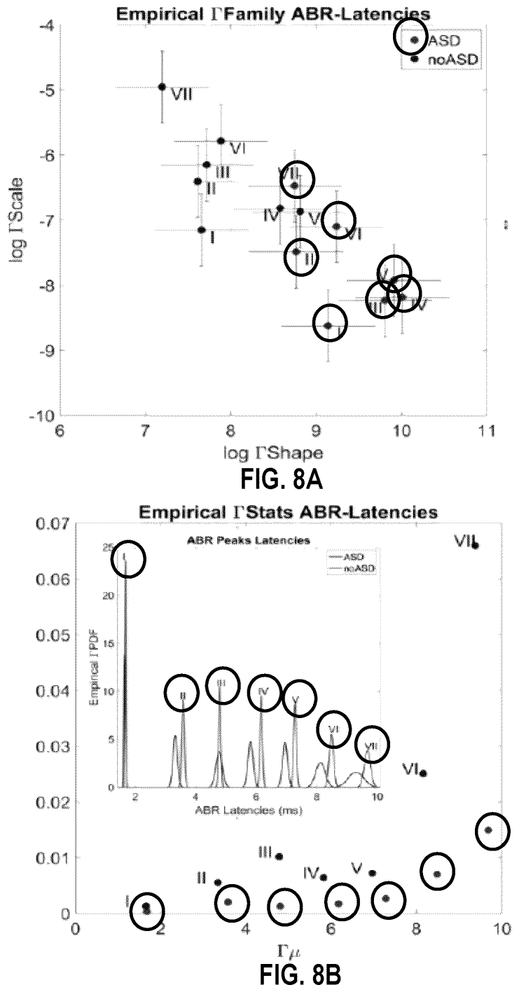

Different families of empirically estimated probability distribution functions (PDFs) of the evoked response potential latencies characterize the asd vs. no-asd neonates across the I-VII vertices. FIG. 8A and FIG. 8B are graphs that illustrate example neurodevelopmental differences in parameters of distributions of latencies of vertices, according to the experimental embodiment. FIG. 8A is log-log Gamma parameter plane spanned by the Gamma shape and scale parameters for each vertex representing a different brainstem region. It shows a complete separation between cohorts. Points represent the PDF's shape and scale (dispersion), with 95% confidence intervals. The encircled asd-neonates cluster in the region of distributions with lower dispersion and more symmetric shape. FIG. 8B plots the empirically estimated Gamma moments (mean versus variance) and further emphasizes the differences in FIG. 8A. The no-asd neonates have broader range of latency variability and faster timings than the encircled asd-neonates. Inset shows empirical PDFs superimposed for both cohorts, the roman numerals for the ASD cohort encircled. These PDFs highlight the shifts in timings (ms) as the evoked response signals propagate along the seven vertices.

It is noted that under equal number of measurements (from bootstrapping), the asd babies have far lower dispersion in their distributions, indicating a much narrower mode of latencies than the non-asd babies. Furthermore, the densities of the distributions of asd babies are shifted to more symmetric shapes, unlike the non-asd babies having more skewed distributions. These distributions with heavy tails emerge from broader ranges of ABR latencies at each of the I-VII vertices.

Pairwise, comparing at each stage the non-asd with the asd stochastic signatures, there is no overlap between these probability distributions representing the two groups. Using the two-sample Kolmogorov-Smirnov test, significant p-values (p<0.01) were found pairwise between the two groups. The frequency histograms from the raw data (not shown) whereby, using bootstrapping techniques to draw from the larger set equal number of points to the smaller set, distributions were estimated with equal number of participants, to compare between cohorts and build a distribution of p-values derived from the comparisons. The pairwise statistical comparison thus obtained with p<0.01 and p<0.05. Furthermore, using the earth mover's distance (EMD), pairwise the pairwise similarity across the 14 frequency histograms (7 vertices from asd vs. 7 vertices from non-asd neonates.) were quantified. The normalized EMD values were used to produce empirical estimates of the PDFs depicted in FIG. 8B inset and offering a view of the systematic shifts (delays) in the asd group.

According to the individual body weight, BW and sex for each group, non-asd females tend to have higher latencies in VI and VII vertices, and overall higher cumulative latencies than non-asd males. However, there were insufficient female neonates in the asd group to have any meaningful trend with statistical power. As such, these results are skewed toward asd males.

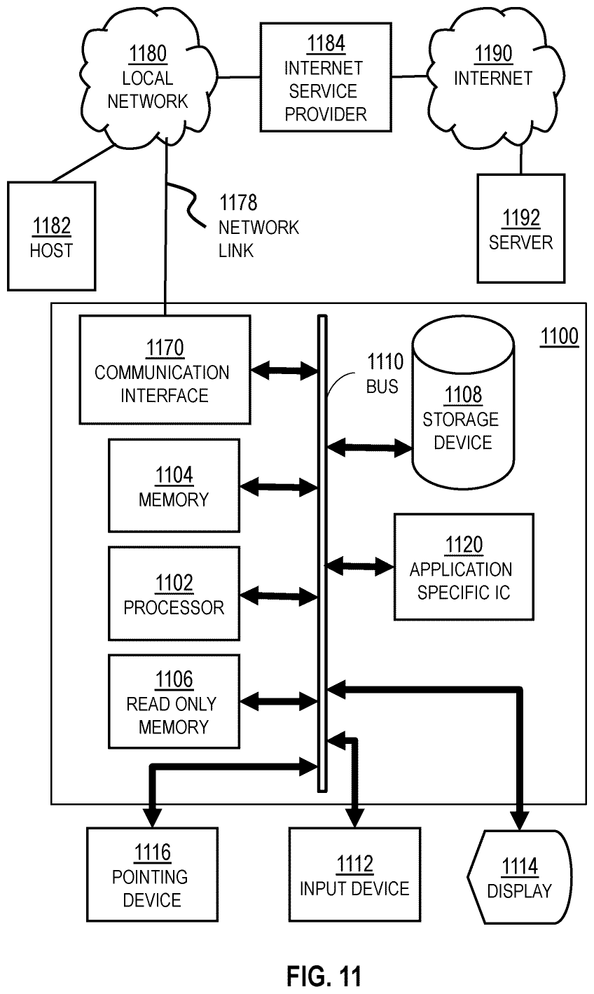

Here, instead of averaging the peaks and smoothing out as noise the fluctuations across the dataset, we obtained the full waveform inclusive of activity prior and after the peak. We examine the variations in the fluctuations of their widths, their prominences, and their full amplitudes (defined in the Methods FIG. 11). Pooling across the autism and non-autism cohorts, we found detectable differences in the empirically estimated Gamma PDFs for each of the features. Most notably, we identified the prominences as the feature revealing the largest differentiation in PDFs between these two cohorts, maximally separating asd from non-asd full-term neonates across the dB levels. This waveform feature also systematically differentiates asd pre-terms from non-asd pre-terms (FIG. 4A shows the separation on the log-log Gamma plane, while FIG. 4B does so for the empirical PDFs. FIG. 4C shows the corresponding empirical Gamma moments along a path connecting the signatures for each dB level's evoked response. We can automatically separate these cohorts along all three dB levels under consideration and distinguish pre-term from full-term neonates according to the asd vs. non-asd subtype. Supplementary FIG. 4 shows results for the peaks' amplitude and widths, while Supplementary FIGS. 5-7 show the stochastic signatures of the range of fluctuations (min and max values) for each of the dB levels. Supplementary FIG. 8A depicts the EMD matrices with entries denoting pairwise similarity in amplitude and inter-peak-interval latencies of the full waveform, while 8B shows the results from non-parametric pairwise comparisons of the differences in peak amplitude values for each subgroup of the 70-75-80 dB-prominences (left) and amplitudes (right). Here the differentiation of three dB levels in full-term vs. pre-term asd neonates are statistically indistinguishable. This means that the fluctuations in the differences in peaks' prominences across the levels cannot separate between pre-term and full-term asd babies. All other pairwise non-parametric comparisons (Wilcoxon rank sum test) yielded statistically significant differences for these three dB levels' differentiation at the 0.05 alpha level.

### 2.2 Micropeak and Primary Peak Prominences

Here, instead of averaging the peaks and smoothing out as noise the fluctuations across the dataset, the full waveform was obtained, inclusive of activity prior and after the primary peak. The variations in the fluctuations were examined, including micropeak widths, prominences, and full amplitudes. Pooling across the autism and non-autism cohorts, detectable differences were found in the empirically estimated Gamma PDFs for each of the features. Most notably, the prominences were identified as the feature revealing the largest differentiation in PDFs between these two cohorts, maximally separating asd from non-asd full-term neonates across the dB levels. This micropeak and primary peak property also systematically differentiated asd pre-terms from non-asd pre-terms.

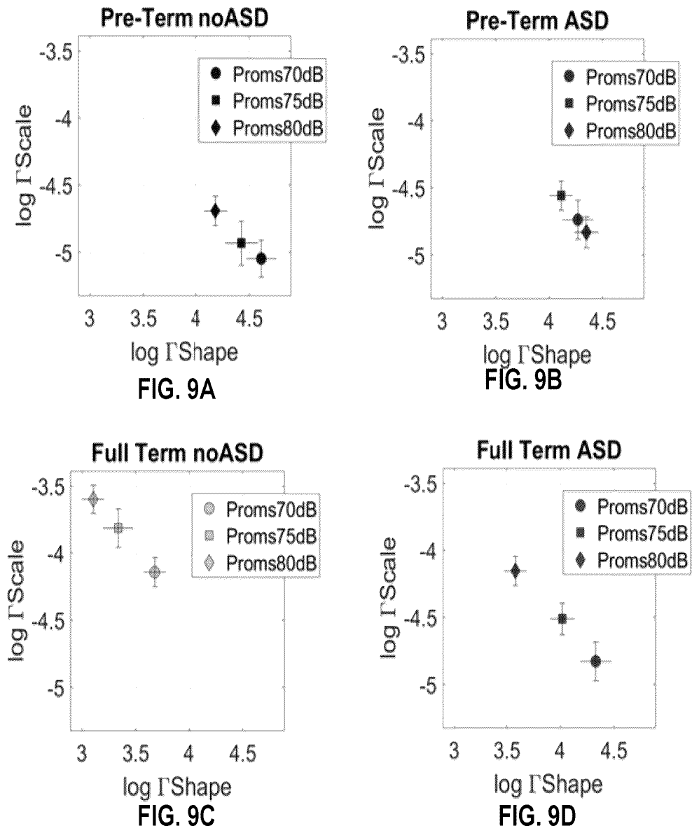

FIG. 9A through FIG. 9D are graphs that illustrate example neurodevelopmental differences in distributions of prominences of peaks in electrical measurements from the ABR test setup, according to the experimental embodiment. These graphs show the Gamma function parameter values for three different auditory volumes 70 dB and 75 dB and 80 dB (with 95% confidence intervals) on the log-log Gamma plane for pre-term no-asd, pre-term asd, full-term no-asd and full term asd, respectively. Fluctuations in peaks' prominences separate dB levels in asd and non-asd pre-term and full-term neonates, and automatically distinguish for each level, the different groups. These levels are different for each of the asd vs. non-asd cases and pre-term vs. full-term cases, with the later showing maximal separability between asd and non-asd neonates

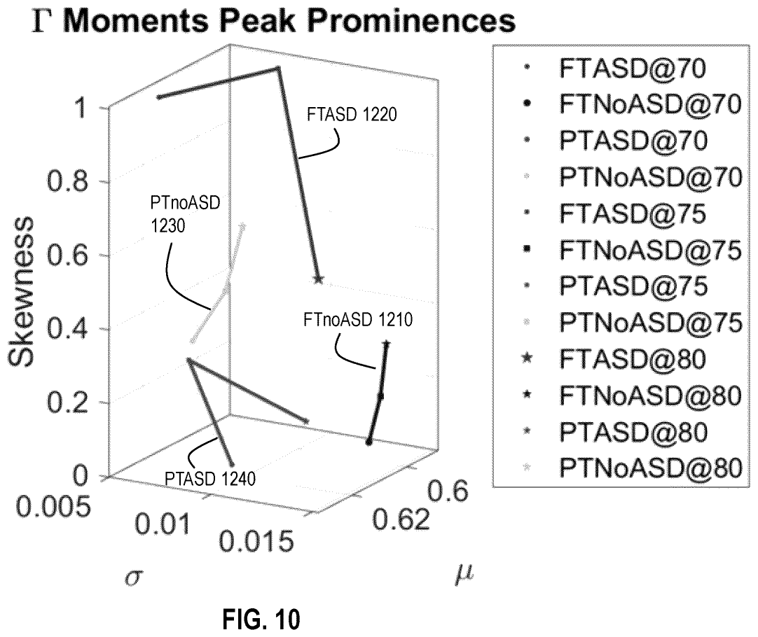

FIG. 10 is a three-dimensional graph that illustrate example clear separation by neurodevelopmental differences in empirical distributions of prominences of peaks in electrical measurements from the ABR test setup, according to the experimental embodiment; Each dimension refers to one of the mean (μ), variance (σ) and skewness of the empirical distributions. Segment lines connect the three auditory volumes for each of the four categories: full-term no-asd 1210, full-term asd 1220, pre-term no-asd 1230, and pre-term asd 1240,

These graphs automatically separate these cohorts along all three dB levels under consideration and distinguish pre-term from full-term neonates according to the asd vs. non-asd subtype. Similar but less dramatic results occur for the peaks' amplitude and widths (not shown). The stochastic signatures of the range of fluctuations (min and max values) for each of the dB levels also distinguish the cohorts. EMD pairwise similarity in amplitude and inter-peak-interval latencies of the full waveform also show separation of the cohorts. The results from non-parametric pairwise comparisons of the differences in peak amplitude values for each subgroup of the 70-75-80 dB-prominences and amplitudes also show separation. Here the differentiation of three dB levels in full-term vs. pre-term asd neonates are statistically indistinguishable. This means that the fluctuations in the differences in peaks' prominences across the levels cannot separate between pre-term and full-term asd babies. All other pairwise non-parametric comparisons (Wilcoxon rank sum test) yielded statistically significant differences for these three dB levels' differentiation at the 0.05 alpha level.

A study of the stochastic ranges between the minimum and maximum shape and scale values also confirmed the differences between groups, along with self-emerging clusters separating asd vs. non-asd individual neonates. This analysis uses a parameter space spanned by the skewness of the distribution of peaks' width along the x-axis, the variance of the distribution of peak's amplitude along the y-axis and included the body weight (BW) at the first visit (panel A), or the estimated gestational age (EGA) at birth (panel B) along the z-axis. Each point in this parameter space represents one baby (personalized stochastic signatures) along each estimated family of probability distributions spanned by the waveforms' features. Automatically emerging clusters of babies reveal that low BW and low estimated gestation age, EGA are not predictors of autism. The cohort of asd neonates had babies across low and high values of both parameters.

### 3. Hardware Overview

FIG. 11 is a block diagram that illustrates a computer system 1100 upon which an embodiment of the invention may be implemented. Computer system 1100 includes a communication mechanism such as a bus 1110 for passing information between other internal and external components of the computer system 1100. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1100, or a portion thereof, constitutes a means for performing one or more steps of one or more methods described herein.

A sequence of binary digits constitutes digital data that is used to represent a number or code for a character. A bus 1110 includes many parallel conductors of information so that information is transferred quickly among devices coupled to the bus 1110. One or more processors 1102 for processing information are coupled with the bus 1110. A processor 1102 performs a set of operations on information. The set of operations include bringing information in from the bus 1110 and placing information on the bus 1110. The set of operations also typically include comparing two or more units of information, shifting positions of units of information, and combining two or more units of information, such as by addition or multiplication. A sequence of operations to be executed by the processor 1102 constitutes computer instructions.

Computer system 1100 also includes a memory 1104 coupled to bus 1110. The memory 1104, such as a random access memory (RAM) or other dynamic storage device, stores information including computer instructions. Dynamic memory allows information stored therein to be changed by the computer system 1100. RAM allows a unit of information stored at a location called a memory address to be stored and retrieved independently of information at neighboring addresses. The memory 1104 is also used by the processor 1102 to store temporary values during execution of computer instructions. The computer system 1100 also includes a read only memory (ROM) 1106 or other static storage device coupled to the bus 1110 for storing static information, including instructions, that is not changed by the computer system 1100. Also coupled to bus 1110 is a non-volatile (persistent) storage device 1108, such as a magnetic disk or optical disk, for storing information, including instructions, that persists even when the computer system 1100 is turned off or otherwise loses power.

Information, including instructions, is provided to the bus 1110 for use by the processor from an external input device 1112, such as a keyboard containing alphanumeric keys operated by a human user, or a sensor. A sensor detects conditions in its vicinity and transforms those detections into signals compatible with the signals used to represent information in computer system 1100. Other external devices coupled to bus 1110, used primarily for interacting with humans, include a display device 1114, such as a cathode ray tube (CRT) or a liquid crystal display (LCD), for presenting images, and a pointing device 1116, such as a mouse or a trackball or cursor direction keys, for controlling a position of a small cursor image presented on the display 1114 and issuing commands associated with graphical elements presented on the display 1114.

In the illustrated embodiment, special purpose hardware, such as an application specific integrated circuit (IC) 1120, is coupled to bus 1110. The special purpose hardware is configured to perform operations not performed by processor 1102 quickly enough for special purposes. Examples of application specific ICs include graphics accelerator cards for generating images for display 1114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1100 also includes one or more instances of a communications interface 1170 coupled to bus 1110. Communication interface 1170 provides a two-way communication coupling to a variety of external devices that operate with their own processors, such as printers, scanners and external disks. In general, the coupling is with a network link 1178 that is connected to a local network 1180 to which a variety of external devices with their own processors are connected. For example, communication interface 1170 may be a parallel port or a serial port or a universal serial bus (USB) port on a personal computer. In some embodiments, communications interface 1170 is an integrated services digital network (ISDN) card or a digital subscriber line (DSL) card or a telephone modem that provides an information communication connection to a corresponding type of telephone line. In some embodiments, a communication interface 1170 is a cable modem that converts signals on bus 1110 into signals for a communication connection over a coaxial cable or into optical signals for a communication connection over a fiber optic cable. As another example, communications interface 1170 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1170 sends and receives electrical, acoustic or electromagnetic signals, including infrared and optical signals, that carry information streams, such as digital data.

The term computer-readable medium is used herein to refer to any medium that participates in providing information to processor 1102, including instructions for execution. Such a medium may take many forms, including, but not limited to, non-volatile media, volatile media and transmission media. Non-volatile media include, for example, optical or magnetic disks, such as storage device 1108. Volatile media include, for example, dynamic memory 1104. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1102, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1102, except for carrier waves and other signals.

Logic encoded in one or more tangible media includes one or both of processor instructions on a computer-readable storage media and special purpose hardware, such as ASIC 1120.

Network link 1178 typically provides information communication through one or more networks to other devices that use or process the information. For example, network link 1178 may provide a connection through local network 1180 to a host computer 1182 or to equipment 1184 operated by an Internet Service Provider (ISP). ISP equipment 1184 in turn provides data communication services through the public, world-wide packet-switching communication network of networks now commonly referred to as the Internet 1190. A computer called a server 1192 connected to the Internet provides a service in response to information received over the Internet. For example, server 1192 provides information representing video data for presentation at display 1114.

The invention is related to the use of computer system 1100 for implementing the techniques described herein. According to one embodiment of the invention, those techniques are performed by computer system 1100 in response to processor 1102 executing one or more sequences of one or more instructions contained in memory 1104. Such instructions, also called software and program code, may be read into memory 1104 from another computer-readable medium such as storage device 1108. Execution of the sequences of instructions contained in memory 1104 causes processor 1102 to perform the method steps described herein. In alternative embodiments, hardware, such as application specific integrated circuit 1120, may be used in place of or in combination with software to implement the invention. Thus, embodiments of the invention are not limited to any specific combination of hardware and software.

The signals transmitted over network link 1178 and other networks through communications interface 1170, carry information to and from computer system 1100. Computer system 1100 can send and receive information, including program code, through the networks 1180, 1190 among others, through network link 1178 and communications interface 1170. In an example using the Internet 1190, a server 1192 transmits program code for a particular application, requested by a message sent from computer 1100, through Internet 1190, ISP equipment 1184, local network 1180 and communications interface 1170. The received code may be executed by processor 1102 as it is received, or may be stored in storage device 1108 or other non-volatile storage for later execution, or both. In this manner, computer system 1100 may obtain application program code in the form of a signal on a carrier wave.

Various forms of computer readable media may be involved in carrying one or more sequence of instructions or data or both to processor 1102 for execution. For example, instructions and data may initially be carried on a magnetic disk of a remote computer such as host 1182. The remote computer loads the instructions and data into its dynamic memory and sends the instructions and data over a telephone line using a modem. A modem local to the computer system 1100 receives the instructions and data on a telephone line and uses an infra-red transmitter to convert the instructions and data to a signal on an infra-red a carrier wave serving as the network link 1178. An infrared detector serving as communications interface 1170 receives the instructions and data carried in the infrared signal and places information representing the instructions and data onto bus 1110. Bus 1110 carries the information to memory 1104 from which processor 1102 retrieves and executes the instructions using some of the data sent with the instructions. The instructions and data received in memory 1104 may optionally be stored on storage device 1108, either before or after execution by the processor 1102.

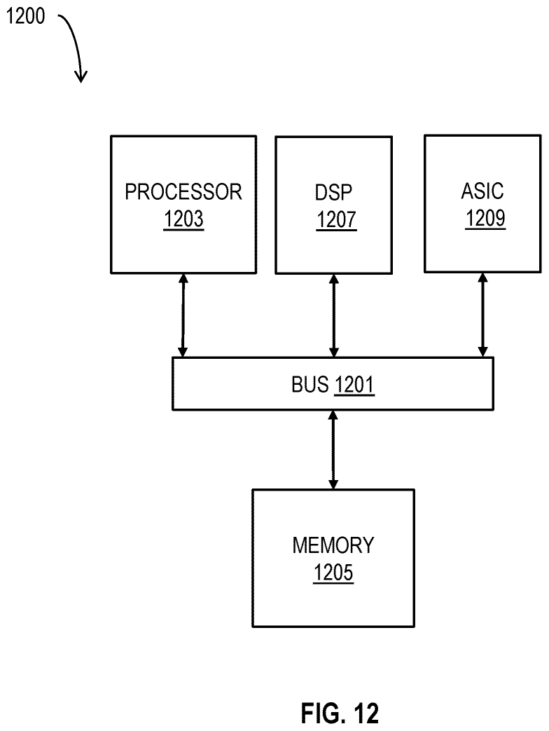

FIG. 12 illustrates a chip set 1200 upon which an embodiment of the invention may be implemented. Chip set 1200 is programmed to perform one or more steps of a method described herein and includes, for instance, the processor and memory components described with respect to FIG. 11 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1200, or a portion thereof, constitutes a means for performing one or more steps of a method described herein.

In one embodiment, the chip set 1200 includes a communication mechanism such as a bus 1201 for passing information among the components of the chip set 1200. A processor 1203 has connectivity to the bus 1201 to execute instructions and process information stored in, for example, a memory 1205. The processor 1203 may include one or more processing cores with each core configured to perform independently. A multi-core processor enables multiprocessing within a single physical package. Examples of a multi-core processor include two, four, eight, or greater numbers of processing cores. Alternatively or in addition, the processor 1203 may include one or more microprocessors configured in tandem via the bus 1201 to enable independent execution of instructions, pipelining, and multithreading. The processor 1203 may also be accompanied with one or more specialized components to perform certain processing functions and tasks such as one or more digital signal processors (DSP) 1207, or one or more application-specific integrated circuits (ASIC) 1209. A DSP 1207 typically is configured to process real-world signals (e.g., sound) in real time independently of the processor 1203. Similarly, an ASIC 1209 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1203 and accompanying components have connectivity to the memory 1205 via the bus 1201. The memory 1205 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1205 also stores the data associated with or generated by the execution of one or more steps of the methods described herein.

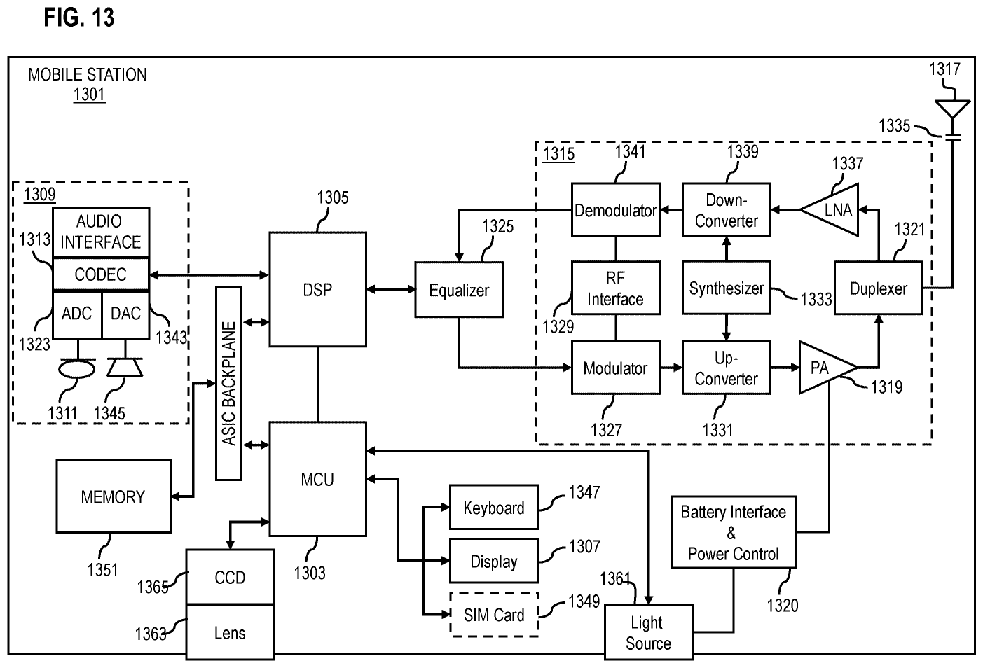

FIG. 13 is a diagram of exemplary components of a mobile terminal 1300 (e.g., cell phone handset) for communications, which is capable of operating in the system of FIG. 1A, according to one embodiment. In some embodiments, mobile terminal 1301, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory (ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1303, a Digital Signal Processor (DSP) 1305, and a receiver/transmitter unit including a microphone gain control unit and a speaker gain control unit. A main display unit 1307 provides a display to the user in support of various applications and mobile terminal functions that perform or support the steps as described herein. The display 1307 includes display circuitry configured to display at least a portion of a user interface of the mobile terminal (e.g., mobile telephone). Additionally, the display 1307 and display circuitry are configured to facilitate user control of at least some functions of the mobile terminal. An audio function circuitry 1309 includes a microphone 1311 and microphone amplifier that amplifies the speech signal output from the microphone 1311. The amplified speech signal output from the microphone 1311 is fed to a coder/decoder (CODEC) 1313.

A radio section 1315 amplifies power and converts frequency in order to communicate with a base station, which is included in a mobile communication system, via antenna 1317. The power amplifier (PA) 1319 and the transmitter/modulation circuitry are operationally responsive to the MCU 1303, with an output from the PA 1319 coupled to the duplexer 1321 or circulator or antenna switch, as known in the art. The PA 1319 also couples to a battery interface and power control unit 1320.

In use, a user of mobile terminal 1301 speaks into the microphone 1311 and his or her voice along with any detected background noise is converted into an analog voltage. The analog voltage is then converted into a digital signal through the Analog to Digital Converter (ADC) 1323. The control unit 1303 routes the digital signal into the DSP 1305 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 1325 for compensation of any frequency-dependent impairments that occur during transmission though the air such as phase and amplitude distortion. After equalizing the bit stream, the modulator 1327 combines the signal with a RF signal generated in the RF interface 1329. The modulator 1327 generates a sine wave by way of frequency or phase modulation. In order to prepare the signal for transmission, an up-converter 1331 combines the sine wave output from the modulator 1327 with another sine wave generated by a synthesizer 1333 to achieve the desired frequency of transmission. The signal is then sent through a PA 1319 to increase the signal to an appropriate power level. In practical systems, the PA 1319 acts as a variable gain amplifier whose gain is controlled by the DSP 1305 from information received from a network base station. The signal is then filtered within the duplexer 1321 and optionally sent to an antenna coupler 1335 to match impedances to provide maximum power transfer. Finally, the signal is transmitted via antenna 1317 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1301 are received via antenna 1317 and immediately amplified by a low noise amplifier (LNA) 1337. A down-converter 1339 lowers the carrier frequency while the demodulator 1341 strips away the RF leaving only a digital bit stream. The signal then goes through the equalizer 1325 and is processed by the DSP 1305. A Digital to Analog Converter (DAC) 1343 converts the signal and the resulting output is transmitted to the user through the speaker 1345, all under control of a Main Control Unit (MCU) 1303 which can be implemented as a Central Processing Unit (CPU) (not shown).

The MCU 1303 receives various signals including input signals from the keyboard 1347. The keyboard 1347 and/or the MCU 1303 in combination with other user input components (e.g., the microphone 1311) comprise a user interface circuitry for managing user input. The MCU 1303 runs a user interface software to facilitate user control of at least some functions of the mobile terminal 1301 as described herein. The MCU 1303 also delivers a display command and a switch command to the display 1307 and to the speech output switching controller, respectively. Further, the MCU 1303 exchanges information with the DSP 1305 and can access an optionally incorporated SIM card 1349 and a memory 1351. In addition, the MCU 1303 executes various control functions required of the terminal. The DSP 1305 may, depending upon the implementation, perform any of a variety of conventional digital processing functions on the voice signals. Additionally, DSP 1305 determines the background noise level of the local environment from the signals detected by microphone 1311 and sets the gain of microphone 1311 to a level selected to compensate for the natural tendency of the user of the mobile terminal 1301.

The CODEC 1313 includes the ADC 1323 and DAC 1343. The memory 1351 stores various data including call incoming tone data and is capable of storing other data including music data received via, e.g., the global Internet. The software module could reside in RAM memory, flash memory, registers, or any other form of writable storage medium known in the art. The memory device 1351 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1349 carries, for instance, important information, such as the cellular phone number, the carrier supplying service, subscription details, and security information. The SIM card 1349 serves primarily to identify the mobile terminal 1301 on a radio network. The card 1349 also contains a memory for storing a personal telephone number registry, text messages, and user specific mobile terminal settings.

In some embodiments, the mobile terminal 1301 includes a digital camera comprising an array of optical detectors, such as charge coupled device (CCD) array 1365. The output of the array is image data that is transferred to the MCU for further processing or storage in the memory 1351 or both. In the illustrated embodiment, the light impinges on the optical array through a lens 1363, such as a pin-hole lens or a material lens made of an optical grade glass or plastic material. In the illustrated embodiment, the mobile terminal 1301 includes a light source 1361, such as a LED to illuminate a subject for capture by the optical array, e.g., CCD 1365. The light source is powered by the battery interface and power control module 1320 and controlled by the MCU 1303 based on instructions stored or loaded into the MCU 1303.

### 4. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10. e.g., 1 to 4.

### 5. REFERENCES

1. A. Starr, R. N. Amlic, W. H. Martin, S. Sanders, Development of auditory function in newborn infants revealed by auditory brainstem potentials. Pediatrics 60, 831-839 (1977).
2. J. S. Baizer, Functional and Neuropathological Evidence for a Role of the Brainstem in Autism. Front Integr Neurosci 15, 748977 (2021).
3. B. Dwornicka, A. Jasienska, W. Smolarz, R. Wawryk, Attempt of Determining the Fetal Reaction to Acoustic Stimulation. Acta Otolaryngol 57, 571-574 (1964).
4. O. Miron et al., Prolonged Auditory Brainstem Response in Universal Hearing Screening of Newborns with Autism Spectrum Disorder. Autism Res 14, 46-52 (2021).
5. J. Sccthapathy, P. Boominathan, A. K. Uppunda, B. Ninan, Auditory brainstem response in very preterm, moderately preterm and late preterm infants. Int J Pediatr Otorhinolaryngol 111, 119-127 (2018).
6. B. Z. Karmel, J. M. Gardner, R. A. Zappulla, C. L. Magnano, E. G. Brown, Brain-stem auditory evoked responses as indicators of early brain insult. Electroencephalogr Clin Neurophysiol 71, 429-442 (1988).
7. A. Scif, C. Shea, S. Schmid, R. A. Stevenson, A Systematic Review of Brainstem Contributions to Autism Spectrum Disorder. Front Integr Neurosci 15, 760116 (2021).
8. C. W. Ponton, J. J. Eggermont, S. G. Coupland, R. Winkelaar, Frequency-specific maturation of the eighth nerve and brain-stem auditory pathway: evidence from derived auditory brain-stem responses (ABRs). J Acoust Soc Am 91, 1576-1586 (1992).
9. Juselius Baghdassarian E, Nilsson Markhed M, Lindström E, Nilsson B M, Lewander T, Auditory brainstem response (ABR) profiling tests as diagnostic support for schizophrenia and adult attention-deficit hyperactivity disorder (ADHD), Acta Neuropsychiatrica DOI: 10.1017/neu.2017.24 (2017)
10. Delgado C F, Simpson E A, Zeng G, Delgado R E, Miron O, Newborn Auditory Brainstem Responses in Children with Developmental Disabilities, J of Autism and Developmental Disorders (2021)
11. Doaa ElMoazena, Ossama Sobhya, Rania Abdoub, HebatAllah AbdelMotaleba, Binaural interaction component of the auditory brainstem response in children with autism spectrum disorder, International Journal of Pediatric Otorhinolaryngology 131 (2020)
12. Burstein O and Geva R (2021) The Brainstem-Informed Autism Framework: Early Life Neurobehavioral Markers. Front. Integr. Neurosci. 15:759614. doi: 10.3389/fnint.2021.759614
13. Sebastian Otto-Meyer, Jennifer Krizman, Travis White-Schwoch, Nina Kraus, Children with autism spectrum disorder have unstable neural responses to sound, Experimental Brain Research (2018)
14. Nicole M. Talge, Melanie Adkins, Paul R. Kileny and Isabella Frownfelter, SYSTEMATIC REVIEW Click-evoked auditory brainstem responses and autism spectrum disorder: a meta-analytic investigation of disorder specificity, European Society for Pediatric Research (2021)
15. Wang X, Zhu M, Samuel O W, Wang X, Zhang H, Yao J, Lu Y, Wang M, Mukhopadhyay S C, Wu W, Chen S and Li G (2020) The Effects of Random Stimulation Rate on Measurements of Auditory Brainstem Response. Front. Hum. Neurosci. 14:78. doi: 10.3389/finhum.2020.00078

The invention claimed is:

1. A method for determining a condition of a subject, the method comprising:
recording auditory brain stem (ABS) electrical signal data in a subject in response to an auditory signal delivered to the subject;
determining first data that indicates a temporal latency for each vertex in a set of one or more vertices in the ABS electrical signal, wherein each vertex indicates a response from a different anatomical location from the cochlea to the auditory cortex of the subject; and
determining a condition of the subject based on a distance of the temporal latency determined for each vertex from a predetermined set of values for temporal latency in a population of control individuals.

2. The method as recited in claim 1, where the population of control individuals includes a plurality of populations of control individuals, each population having a different set of values for temporal latency and associated with a different neurodevelopmental condition.

3. The method as recited in claim 2, where the plurality of populations of control individuals, includes first population associated with normal development and different second population associated with autism spectrum disorder (ASD).

4. The method as recited in claim 1, where the temporal latency is measured from a time when the auditory signal is delivered to the subject.

5. The method as recited in claim 1, where the temporal latency is measured from a time of a reference vertex of the set of vertices to a particular vertex in the set of one or more vertices.

6. The method as recited in claim 5, where the reference vertex is the temporally first vertex to occur in the electrical signal associated with the cochlea of the subject.

7. The method as recited in claim 5, where the reference vertex is the vertex that temporally immediately precedes the particular vertex.

8. The method as recited in claim 2, wherein:
said recording auditory brain stem (ABS) electrical signal data in the subject includes recording auditory brain stem (ABS) electrical signal data in the subject in response to a plurality of auditory signals delivered to the subject;
the first data indicates a distribution of latencies for each vertex over the plurality of auditory signals delivered to the subject; and
the distance is a distance between the distribution of latencies of the subject to the distributions of latencies in the plurality of populations of control individuals.

9. The method as recited in claim 8, where the distance is an Earthmover's distance between distributions.

10. The method as recited in claim 2 where the different set of values for temporal latency are indicated by a different set of values for parameters of a probability density function.

11. The method as recited in claim 10, where the probability density function is a Gamma Function and the parameters of the Gamma Function include scale and shape.

12. The method as recited in claim 10, where the parameters of the probability density function include mean, variance and skewness.

13. A method for determining a condition of a subject, the method comprising:
recording auditory brain stem (ABS) electrical signal data in a subject in response to an auditory signal delivered to the subject;
determining first data that indicates a property of micropeaks in the ABS electrical signal, wherein micropeaks occur in a first portion of the ABS electrical signal away from a different second portion that includes a set of one or more vertices, wherein each vertex indicates a response from a different anatomical location from the cochlea to the auditory cortex of the subject; and
determining a condition of the subject based on a distance of the property of the micropeaks from a predetermined set of values for the property of the micropeaks in a population of control individuals.

14. The method as recited in claim 13, where the population of control individuals includes a plurality of populations of control individuals, each population having a different set of values for the property of the micropeaks and associated with a different neurodevelopmental condition.

15. The method as recited in claim 14, where the plurality of populations of control individuals, includes first population associated with normal development and different second population associated with autism spectrum disorder (ASD).

16. The method as recited in claim 13, where the property of the micropeaks includes a prominence for each micropeak.

17. The method as recited in claim 13, where the property of the micropeaks includes a width for each micropeak.

18. The method as recited in claim 13, where the property of the micropeaks includes an amplitude for each micropeak.

19. The method as recited in claim 14, wherein:
the first data indicates a distribution of the property for each micropeak over a plurality of micropeaks; and
the distance is a distance between the distribution of the property of the subject to the distributions of the property in the plurality of populations of control individuals.

20. The method as recited in claim 19, where the distance is an Earthmover's distance between distributions.

21. The method as recited in claim 13 where the different set of values for the property of the micropeaks are indicated by a different set of values for parameters of a probability density function for the property.

22. The method as recited in claim 21, where the probability density function is a Gamma Function and the parameters of the Gamma Function include scale and shape.

23. The method as recited in 21, where the parameters of the probability density function include mean, variance and skewness.

24. A non-transitory computer-readable medium carrying one or more sequences of instructions for determining a condition of a subject, wherein execution of the one or more sequences of instructions by one or more processors causes the one or more processors to at least:
record auditory brain stem (ABS) electrical signal data in a subject in response to an auditory signal delivered to the subject;
determine first data that indicates a temporal latency for each vertex in a set of one or more vertices in the ABS electrical signal, wherein each vertex indicates a response from a different anatomical location from the cochlea to the auditory cortex of the subject; and
determine a condition of the subject based on a distance of the temporal latency determined for each vertex from a predetermined set of values for temporal latency in a population of control individuals.

25. An apparatus for determining a condition of a subject, the apparatus comprising:
at least one processor; and
at least one memory including one or more sequences of instructions,
the at least one memory and the one or more sequences of instructions configured to, with the at least one processor, cause the apparatus to:
record auditory brain stem (ABS) electrical signal data in a subject in response to an auditory signal delivered to the subject;
determine first data that indicates a temporal latency for each vertex in a set of one or more vertices in the ABS electrical signal, wherein each vertex indicates a response from a different anatomical location from the cochlea to the auditory cortex of the subject; and
determine a condition of the subject based on a distance of the temporal latency determined for each vertex from a predetermined set of values for temporal latency in a population of control individuals.

26. A system for determining a condition of a subject, the system comprising:
auditory brainstem response test equipment;
at least one processor; and
at least one memory including one or more sequences of instructions,
the at least one memory and the one or more sequences of instructions configured to, with the at least one processor, cause the apparatus to:
record auditory brain stem (ABS) electrical signal data in a subject in response to an auditory signal delivered to the subject by the test equipment;
determine first data that indicates a temporal latency for each vertex in a set of one or more vertices in the ABS electrical signal, wherein each vertex indicates a response from a different anatomical location from the cochlea to the auditory cortex of the subject; and
determine a condition of the subject based on a distance of the temporal latency determined for each vertex from a predetermined set of values for temporal latency in a population of control individuals.

27. A non-transitory computer-readable medium carrying one or more sequences of instructions for determining a condition of a subject, wherein execution of the one or more sequences of instructions by one or more processors causes the one or more processors to:
record auditory brain stem (ABS) electrical signal data in a subject in response to an auditory signal delivered to the subject;
determine first data that indicates a property of micropeaks in the ABS electrical signal, wherein micropeaks occur in a first portion of the ABS electrical signal away from a different second portion that includes a set of one or more vertices, wherein each vertex indicates a response from a different anatomical location from the cochlea to the auditory cortex of the subject; and
determine a condition of the subject based on a distance of the property of the micropeaks from a predetermined set of values for the property of the micropeaks in a population of control individuals.

28. An apparatus for determining a condition of a subject, the apparatus comprising:
at least one processor; and
at least one memory including one or more sequences of instructions,
the at least one memory and the one or more sequences of instructions configured to, with the at least one processor, cause the apparatus to:
record auditory brain stem (ABS) electrical signal data in a subject in response to an auditory signal delivered to the subject;
determine first data that indicates a property of micropeaks in the ABS electrical signal, wherein micropeaks occur in a first portion of the ABS electrical signal away from a different second portion that includes a set of one or more vertices, wherein each vertex indicates a response from a different anatomical location from the cochlea to the auditory cortex of the subject; and
determine a condition of the subject based on a distance of the property of the micropeaks from a predetermined set of values for the property of the micropeaks in a population of control individuals.

29. A system for determining a condition of a subject, the system comprising:
auditory brainstem response test equipment;
at least one processor; and
at least one memory including one or more sequences of instructions,
the at least one memory and the one or more sequences of instructions configured to, with the at least one processor, cause the apparatus to:
record auditory brain stem (ABS) electrical signal data in a subject in response to an auditory signal delivered to the subject by the test equipment;
determine first data that indicates a property of micropeaks in the ABS electrical signal, wherein micropeaks occur in a first portion of the ABS electrical signal away from a different second portion that includes a set of one or more vertices, wherein each vertex indicates a response from a different anatomical location from the cochlea to the auditory cortex of the subject; and
determine a condition of the subject based on a distance of the property of the micropeaks from a predetermined set of values for the property of the micropeaks in a population of control individuals.
